(19) United States
(12) Patent Application Publication
(10) Pub. No.: US 2004/0102361 A1
(43) Pub. Date: May 27, 2004

(54) Pharmaceutical composition for the treatment of pulmonary arterial hypertension

(76) Inventor: Frederic Bodin, San Francisco, CA (US)

Correspondence Address: JONES DAY, 222 EAST 41ST STREET, NEW YORK, NY, 10017, US

(21) Appl. No.: 10/306,434

(22) Filed: Nov. 27, 2002

(51) Int. Cl. A61K 31/557

(52) U.S. Cl. 514/2; 514/573

(57) ABSTRACT

The invention relates to pharmaceutical compositions for the treatment of pulmonary arterial hypertension comprising a prostacyclin or a prostacyclin analogue, preferably epoprostenol, and an endothelin receptor antagonist, preferably bosentan. The invention further provides methods for treating a subject suffering from pulmonary arterial hypertension using the compositions of the invention. The concomitant administration of prostacyclin or a prostacyclin analogue and an endothelin receptor antagonist not only increases the efficacy compared to administration of each alone but also reduces the side effects associated with prostacyclin or prostacyclin analogues.

The invention relates to pharmaceutical compositions for the treatment of pulmonary arterial hypertension comprising a prostacyclin or a prostacyclin analogue and an endothelin receptor antagonist, characterized in that the side effects of the prostacyclin or the prostacyclin analogue are greatly reduced by the concomitant administration of the prostacyclin or the prostacyclin analogue and the endothelin receptor antagonist.

Pulmonary hypertension is a disease defined by a progressive elevation of pulmonary artery pressure and pulmonary vascular resistance, leading to right ventricular failure and death. Pulmonary hypertension is associated with endothelial dysfunction, characterized by a decreased expression of the vasodilators nitric oxide and prostacyclin, and by an increased expression of the growth factor and vasoconstrictive substance endothelin-1 and its receptors.

Prostacyclin and prostacyclin analogues such as epoprostenol, treprostinil, iloprost, beraprost significantly improve hemodynamic parameters and clinical symptoms in patients with pulmonary arterial hypertension. The major mechanism of action of prostacyclin and prostacyclin analogues is vasodilation, whereas improvement in pulmonary vascular hypertrophy and inhibition of platelet aggregation may also play a role. However, the use of prostacyclin or prostacyclin analogues is associated with a number of side effects such as jaw pain, headaches, flushing, tachycardia and systemic hypotension.

Endothelin receptor antagonists such as bosentan (4-tert-butyl-N-[6-(2-hydroxyethoxy)-5-(2-methoxy-phenoxy)-2,2′-bipyrimidin-4-yl]-benzene-sulfonamide) are also efficacious in the treatment of pulmonary arterial hypertension. Bosentan improves hemodynamic parameters (cardiac index, pulmonary artery pressure, pulmonary vascular resistance), increases exercise capacity, improves WHO functional class, and decreases the rate of clinical worsening in patients with pulmonary arterial hypertension. Bosentan does not significantly modify heart rate or mean arterial blood pressure in patients with pulmonary arterial hypertension.

The mechanism of action of endothelin receptor antagonists is competitive antagonism of the binding of ET-1 on ET receptors, thereby decreasing pulmonary vasoconstriction and vascular remodeling. Endothelin receptor antagonists, by their inhibition of the endothelin system, further inhibit the activation of other neurohormonal systems, and in particular reduce sympathetic nerve activity, decrease catecholamine concentrations and blunt reactive tachycardia in response to a decrease in blood pressure.

The combination of bosentan and a prostacyclin, especially epoprostenol (5Z, 9α, 111α, 13E, 15)-6,9-epoxy-11,15-dihydroxyprosta-5,13-dien-1-oic acid, sodium salt (cf., U.S. Pat. No. 4,539,333), has been evaluated in a clinical study. The authors of Am J Respir Crit Care Med, 165:1209-1216, 2002, who were running on behalf of Actelion Pharmaceuticals Ltd the clinical trials of the combination of these two drugs, speculated that this combination may have additional efficacy. The outcome of the trial was, however, entirely unexpected.

The basis of the present application is the unexpected finding in the clinical trial initiated and supervised by Actelion Pharmaceuticals Ltd that the combination of bosentan with epoprostenol not only has additional efficacy, but also decreases the risk of side effects related to epoprostenol considerably. Indeed, in patients treated with bosentan and epoprostenol, there were fewer reported cases of jaw pain, headaches and systemic hypotension, a lesser decrease in blood pressure and a lesser increase in heart rate as compared to patients treated with epoprostenol alone. This may allow to combine two efficacious treatments with a better safety profile as compared to a prostacyclin or prostacyclin analogue alone, and also to decrease the risk of exaggeration of side effects upon stopping the administration of a prostacyclin or prostacyclin analogue.

Especially preferred are pharmaceutical compositions for the treatment of pulmonary arterial hypertension comprising epoprostenol and the other bosentan, characterized in that the side effects of epoprostenol are strongly reduced by the concomitant administration of epoprostenol and bosentan or by preferably administering bosentan within a time frame of ninety six hours after epoprostenol has been administered.

The use of the pharmaceutical compositions mentioned above is leading to an improvement of the patients as compared to the use of prostacyclin or prostacyclin analogues alone. Therefore, a new method of treating patients with epoprostenol and bosentan has been established or in a more general manner a method of treating patients suffering from pulmonary arterial hypertension with a prostacyclin or a prostacyclin analogue followed by administering an endothelin antagonist has been found.

The dose of the prostacyclin may vary between about 1 ng/kg/min and about 250 ng/kg/min depending on the length it has been already administered. Preferably, the dosage is between about 1 ng/kg/min and about 100 ng/kg/min, more preferably between about 1 ng/kg/min and about 50 ng/kg/min, and most preferably about 2 ng/kg/min. With increasing time, the dose is increased. A preferred use of the pharmaceutical compositions resides in administering for two days 2 ng/kg/min, then increasing every two weeks the dose by 2 ng/kg/min up to the preferred target dose of 14±2 ng/kg/min. After the first two days of treatment with prostacyclin or an analogue thereof, bosentan is administered twice a day at a dose of either 62.5 mg or 125 mg. The resulting advantages and benefits are disclosed in the following description of a clinical trial, which illustrates the invention.

In general a dose range for the prostacyclin of about 0.01 to about 200 mg per kilogram body weight, conveniently about 0.01 to about 10 mg per kilogram body weight, is used. The dose range for the endothelin antagonist may be between about 0.01 mg to about 10 mg per kilogram body weight, conveniently about 0.5 mg to about 3.0 mg per kilogram body weight. The preparation of a pharmaceutical composition containing a prostacyclin has been described in U.S. Pat. No. 4,539,333, which is incorporated by reference in its entirety.

The preparation of a pharmaceutical composition containing an endothelin antagonist, e.g. bosentan, is described in U.S. Pat. No. 5,292,740 and is also incorporated by reference in its entirety.

Summary of a Clinical Trial (Protocol AC-052-355)

This was a double-blind, randomized, placebo-controlled study to assess the effects of bosentan on hemodynamics, safety and tolerability in patients with severe pulmonary arterial hypertension when combined with the initiation of epoprostenol therapy.

Study design: The duration of the study was 16 weeks, 2:1 bosentan:placebo randomization. All patients received the starting epoprostenol dose of 2 ng/kg/min for 2 days, then they were randomized to receive either bosentan or placebo. Every 2 weeks thereafter, epoprostenol was increased by 2 ng/kg/min up to the target dose of 14±2 ng/kg/min by week 16. All patients received epoprostenol together with either bosentan or placebo for 16 weeks. Hemodynamic assessments were performed at baseline (prior to start of therapy) and again after 16 weeks of therapy. Safety monitoring was performed throughout the study duration.

Primary endpoint: Percent change from baseline in total pulmonary resistance (TPR) to week 16. The sample size was estimated for an expected mean difference in TPR of 28%.

Secondary endpoints: Changes from baseline to week 16 in CI (Cardiac Index), PVR (Pulmonary Vascular Resistance), mRAP (mean pulmonary arterial pressure), and mRAP (mean right atrial pressure); changes from baseline to week 16 in walk distance (6-min walk test), dyspnea fatigue rating, WHO functional class; and safety and tolerability.

Results:

Thirty-three patients entered the study: 11 patients received epoprostenol alone, while 22 patients received bosentan with epoprostenol. The results are summarized in Table 1 below.

Hemodynamic Efficacy:

The results show a positive trend toward an improvement in hemodynamic parameters, specifically increase in Cardiac Index (CI) and decreases in Total Pulmonary Resistance (TPR), mean Pulmonary Arterial Pressure (mPAP) and mean Right Atrial Pressure (mRAP), in patients who received bosentan with epoprostenol compared to those who received epoprostenol alone. The Hemodynamic efficacy data are summarized in Table 1 below.

TABLE I
Hemodynamic Efficacy Results:

| | TPR | | CI | | mPAP | | mRAP | |
|---|---|---|---|---|---|---|---|---|
| | (dyn*sec/cm⁵) | | (L/min/m²) | | mmHg | | mmHg | |
| Epoprostenol Alone | | | | | | | | |
| Baseline | 1628 ± | 511 | 1.75 ± | 0.66 | 60.9 ± | 9.7 | 11.9 ± | 7.4 |
| | | | | | mmHg | | | |
| % mean change | −22.6 ± | 20.4% | 37.9 ± | 44.1% | N/A | | N/A | |
| Absolute change | −386 ± | 403 | 0.57 ± | 0.68 | −1.6 ± | 7.2 | 0.3 ± | 4.4 |
| | | | | | mmHg | | mmHg | |
| % median change | (−14.3%) | | (30.0%) | | N/A | | N/A | |
| Bosentan & epoprostenol | | | | | | | | |
| Baseline | 1697 ± | 666 | 1.73 ± | 0.43 | 59.2 ± | 13.4 | 11.9 ± | 5.3 |
| | | | | | mmHg | | | |
| % mean change | −36.3 ± | 20.1% | 48.7 ± | 51.4% | −9.0 ± | 19.8% | N/A | |
| Absolute change | −681 ± | 569 | 0.75 ± | 0.64 | −6.7 ± | 12.8 | −1.9 ± | 6.7 |

| | | | | |
|---|---|---|---|---|
| | | | mmHg | |
| % median change | (−36.8%) | (39.7%) | N/A | N/A |

In Table 1 the results are expressed as changes from baseline at week 16. Mean±SD (standard deviation) and median change (in brackets).

Safety and Tolerability

Vital Signs

Concomitant administration of bosentan with epoprostenol reduced the fall in systolic blood pressure resulting from monotherapy with epoprostenol and also prevented the epoprostenol-induced increase in heart rate.

TABLE 2
Mean and median changes from baseline to week 16:

| | | Bosentan & |
|---|---|---|
| | Epoprostenol alone | epoprostenol |
| Systolic blood pressure (mmHg) | −9.8 ± 10.9% | −3.9 ± 14.3% |
| Heart Rate (bpm) | | |
| Mean ± standard deviation | 6.6 ± 11.9% | −0.5 ± 13.9% |
| median | 7.0 | −3.0 |

Symptomatic Adverse Events:

The frequency of epoprostenol-related side effects (jaw pain, flushing, headache and hypotension) was lower in patients receiving the combined bosentan and epoprostenol therapy compared to those receiving epoprostenol alone (Table 3).

TABLE 3
Summary of treatment emergent adverse events

| | Epoprostenol alone | | Bosentan + Epoprostenol | |
|---|---|---|---|---|
| Adverse event | No. | % | No. | % |
| PAIN IN JAW | 10 | 90.9% | 13 | 59.1% |
| FLUSHING | 5 | 45.5% | 6 | 27.3% |
| HEADACHE | 4 | 36.4% | 6 | 27.3% |
| HYPOTENSION | 2 | 18.2% | — | |

The combination of bosentan with epoprostenol or any other prostacyclin analogues leads to an additional efficacy and less prostanoid-related side effects.

Results: In addition to the improvement in efficacy, there were less side effects related to prostacyclin analogues when bosentan is added to a prostacyclin analogue.

What is claimed is:

1. A pharmaceutical composition for the treatment of pulmonary arterial hypertension comprising: (i) an effective amount of a prostacyclin or a prostacyclin analogue; and (ii) an effective amount of endothelin receptor antagonist.

2. The pharmaceutical composition of claim 1, wherein the prostacyclin is epoprostenol.

3. The pharmaceutical composition of claim 2, wherein the endothelin receptor antagonist is bosentan.

4. The pharmaceutical composition of claim 1, 2, or 3, wherein (i) and (ii) are pre-mixed.

5. The pharmaceutical composition of claim 1, 2, or 3, wherein (i) and (ii) are not pre-mixed.

6. A method of treating a subject having pulmonary arterial hypertension comprising administering to the subject an effective amount of a prostacyclin or a prostacyclin analogue in combination with an effective amount of endothelin receptor antagonist.

7. The method of claim 6, wherein the prostacyclin is epoprostenol.

8. The method of claim 6, wherein the endothelin receptor antagonist is bosentan.

9. The method of claim 7, wherein the endothelin receptor antagonist is bosentan.

10. The method of claim 6, 7, 8, or 9, wherein the prostacyclin is administered at a dose between about 1 ng/kg/min and about 25 ng/kg/min and the endothelin receptor antagonist at a dose between about 0.01 mg and about 10 mg per kilogram body weight.

11. The method of claim 10, wherein the prostacyclin is administered at a dose between about 2 ng/kg/min and about 14 ng/kg/min and the endothelin receptor antagonist at a dose between about 0.5 mg and about 3.0 mg per kilogram body weight.

12. The method of claim 9, wherein epoprostenol is administered to the subject at a dose of 2 ng/kg/min for two days followed by a dose increased by 2 ng/kg/min every two weeks up to the target dose of 14±2 ng/kg/min and, in addition, after the first two days, bosentan is administered at a dose of 62.5 mg or 125 mg twice a day.
